(19) United States
(12) Patent Application Publication
(10) Pub. No.: US 2023/0329534 A1
(43) Pub. Date: Oct. 19, 2023

(54) MEDICAL DEVICES AND RELATED SYSTEMS AND METHODS

(71) Applicants: Boston Scientific Medical Device Limited, Galway (IE); Boston Scientific Scimed, Inc., Maple Grove, MN (US)

(72) Inventors: Aditya DHANOTIYA, Indore (IN); Nabarun BHOWMICK, Kolkata (IN); Shrikant Vasant RAUT, Mumbai (IN); James WELDON, Newton, MA (US)

(73) Assignees: Boston Scientific Medical Device Limited, Galway (IE); Boston Scientific Scimed, Inc., Maple Grove, MN (US)

(21) Appl. No.: 18/302,127

(22) Filed: Apr. 18, 2023

Related U.S. Application Data
(60) Provisional application No. 63/363,203, filed on Apr. 19, 2022.

(51) Int. Cl. A61B 1/005

(52) U.S. Cl. CPC A61B 1/0052; A61M 25/0136

(57) ABSTRACT

According to one aspect, a connector assembly for a medical device may comprise a sleeve configured to fixedly couple to a shaft of the medical device and rotatably couple to a handle of the medical device; a collar positioned around the sleeve and configured to couple to the sleeve such that collar (i) may move in a proximal and distal direction relative to sleeve and (ii) may rotate sleeve about a central longitudinal axis when the collar is rotated about the central longitudinal axis; and a biasing member. The connector assembly may be configured to transition between a neutral position and an engaged position.

### CROSS-REFERENCE TO RELATED APPLICATIONS

This application claims the benefit of priority of U.S. Provisional Patent Application No. 63/363,203, filed Apr. 19, 2022, the entirety of which is incorporated herein by reference.

### TECHNICAL FIELD

Various aspects of this disclosure relate generally to handles, connectors, and shafts of medical devices. More specifically, embodiments of this disclosure relate to rotatable connector assemblies for use with a handle of an endoscope or other medical device, among other aspects.

### BACKGROUND

During endoscopic procedures, a medical professional operating an endoscope often wraps his/her entire palm around a grip or handle portion of the device. Various actuators on the handle of the endoscope require the medical professional to contort his/her hands and move the handle frequently and for prolonged periods of time during a procedure, which can cause strain, or even an injury. In some cases, actuation of different scope controls, like knobs or an elevator, may result in the shaft of the endoscope becoming twisted and may exert excessive force on the handle of the endoscope, which may result in strain in the medical professional's hand. Endoscope operators can experience wrist and hand discomfort resulting from holding and manipulating the endoscope's handle, in some cases due to force applied by a twisted proximal portion of the shaft of the endoscope as the handle is moved in the operating room. In some cases, for example, medical professionals may experience symptoms similar to those of Carpal Tunnel Syndrome or tendonitis. When a medical professional experiences fatigue or other pain in the fingers, hand, or wrist, the medical professional may shift from a primary grip position to a secondary grip position that may be a less powerful grip than the primary grip position, such as shifting from a four finger grip to a three finger grip. Repeatedly bending and maneuvering of the wrist to adjust positioning of the endoscope can increase fatigue or other pain.

When a medical professional repeatedly readjusts his or her hand grip in between procedure tasks, the procedure may be prolonged and procedural tasks may be more difficult. Depending on the strength of a medical professional's hand and arm, force applied to the handle by a twisted proximal portion of the endoscope may increase the number of readjustments of the professional's hand during a procedure.

### SUMMARY

Aspects of the disclosure relate to, among other things, systems, devices, and methods for alleviating force applied to a medical device's handle by a twisted distal portion of the device. The systems, devices, and methods of this disclosure may facilitate a user's hand grip on an endoscope or other medical device. Endoscopes and other medical devices with a handle that may rotate relative to a proximal shaft portion may help address user hand fatigue or strain, may help reduce the need for hand grip readjustments, and may help address other issues. Each of the aspects disclosed herein may include one or more of the features described in connection with any of the other disclosed aspects.

According to one aspect, a connector assembly for a medical device may comprise a sleeve configured to fixedly couple to a shaft of the medical device and rotatably couple to a handle of the medical device; a collar positioned around the sleeve and configured to couple to the sleeve such that collar (i) may move in a proximal and distal direction relative to sleeve and (ii) may rotate sleeve about a central longitudinal axis when the collar is rotated about the central longitudinal axis; and a biasing member. The connector assembly may be configured to transition between a neutral position and an engaged position.

In other aspects the connector assembly may include one or more of the following features. The knob portion and the collar cannot rotate relative to the handle in the neutral position; and the knob portion and the collar can rotate relative to the handle in the engaged position. The connector assembly may further comprise a knob portion positioned around the collar; and the knob portion may include ribs. The sleeve may include at least one protrusion at a proximal portion of the sleeve, and the at least one protrusion is configured to be received in a recess of the handle. The biasing member may be a spring. The connector assembly may further comprise a tube extending through the collar. The sleeve may include a series of longitudinal ribs and the collar may include a series of longitudinal recesses configured to receive the longitudinal ribs. The longitudinal ribs may be configured to move proximally and distally through the longitudinal recesses. The collar may include a protrusion extending circumferentially around a central longitudinal axis of the collar. The knob portion may include a recess extending circumferentially around a radially-inward facing surface of the knob portion, and the recess may be configured to receive the protrusion. Each of the collar, the knob portion, and the sleeve may be approximately cylindrical and includes a respective central longitudinal lumen. The collar may include a series of teeth extending proximally from a proximal end portion of the collar. The series of teeth may be configured to be received by a complimentary series of teeth of the handle. The series of teeth of the collar may be received by the complimentary series of teeth of the handle in the neutral position; and the series of teeth of the collar may be spaced from the complimentary series of teeth of the handle in the engaged position. The collar may be fixedly coupled to the knob portion.

In other aspects, a medical device may comprise a handle body; a shaft extending longitudinally to a distal tip; and a connector assembly. The connector assembly may comprise a sleeve fixedly coupled to the shaft and rotatably coupled to a handle body; a collar positioned around the sleeve and coupled to the sleeve such that collar (i) is movable in a proximal and distal direction relative to sleeve and (ii) is rotatable about a central longitudinal axis of the medical device during rotation of the collar about the central longitudinal axis; a knob portion positioned around the collar; and a biasing member. The connector assembly may be configured to transition between a neutral position and an engaged position.

In other aspects the connector assembly may include one or more of the following features. The collar may include a series of teeth extending proximally from a proximal end portion of the collar; and the series of teeth may be configured to be received by a complimentary series of teeth of the handle body. The sleeve may include protrusions at a proximal portion of the sleeve, and the protrusions are configured to be received in recesses of the handle body.

In other aspects, a method of operating a medical device that includes a handle and a shaft extending longitudinally from the handle, the method may comprise: moving a knob portion of a connector assembly distally and compressing a biasing member of the connector assembly; rotating the knob portion about a central longitudinal axis of the knob portion relative to the handle; and releasing the knob portion to fixedly couple the shaft to the handle. In some examples, moving the knob portion of the connector assembly distally may include moving teeth of a collar of the connector assembly away from teeth at a proximal end portion of the handle.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary aspects of this disclosure and together with the description, serve to explain the principles of the disclosure.

FIGS. 1A and 1B are perspective views of an exemplary endoscope, according to aspects of this disclosure.

FIGS. 2A and 2B are perspective views of a user's hand holding an endoscope handle (such as a handle of the endoscope of FIGS. 1A and 1B), according to aspects of this disclosure.

FIG. 3 is a side view of a portion of an endoscope including a rotatable connector assembly, according to aspects of this disclosure.

FIG. 4 is a side, cross-sectional view of the rotatable connector assembly of FIG. 3, according to aspects of this disclosure.

FIG. 5 is a perspective view of the rotatable connector assembly of FIGS. 3 and 4 with a portion removed to expose interior components, according to aspects of this disclosure.

FIG. 6 is a side, partial cross-sectional view of a proximal portion of the endoscope of FIG. 3, according to aspects of this disclosure.

FIG. 7 is a side, partial cross-sectional view of the rotatable connector assembly of FIG. 3, according to aspects of this disclosure.

FIGS. 8A-8D are partial cross-sectional views of the rotatable connector assembly of FIG. 3, according to aspects of this disclosure.

### DETAILED DESCRIPTION

Reference will now be made in detail to aspects of this disclosure, examples of which are illustrated in the accompanying drawings. Wherever possible, the same or similar reference numbers will be used through the drawings to refer to the same or like parts. The term “distal” refers to a portion farthest away from a user when introducing a device into a patient. By contrast, the term “proximal” refers to a portion closest to the user when placing the device into the patient. Throughout the figures included in this application, arrows labeled “P” and “D” are used to show the proximal and distal directions in the figure. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Further, relative terms such as, for example, “about,” “substantially,” “approximately,” etc., are used to indicate a possible variation of ±10% in a stated numeric value or range.

Embodiments of this disclosure may improve a user's ability to grip, manipulate, and otherwise apply force to a handle, and actuators of the handle, of a medical device, such as an endoscope, during a medical procedure and, as non-limiting exemplary benefits, help reduce the need to reposition a user's hand during a procedure and reduce strain to a user's hand from excessive movement, among other aspects. Embodiments of this disclosure may also facilitate rotation of a medical device shaft relative to a handle of the medical device.

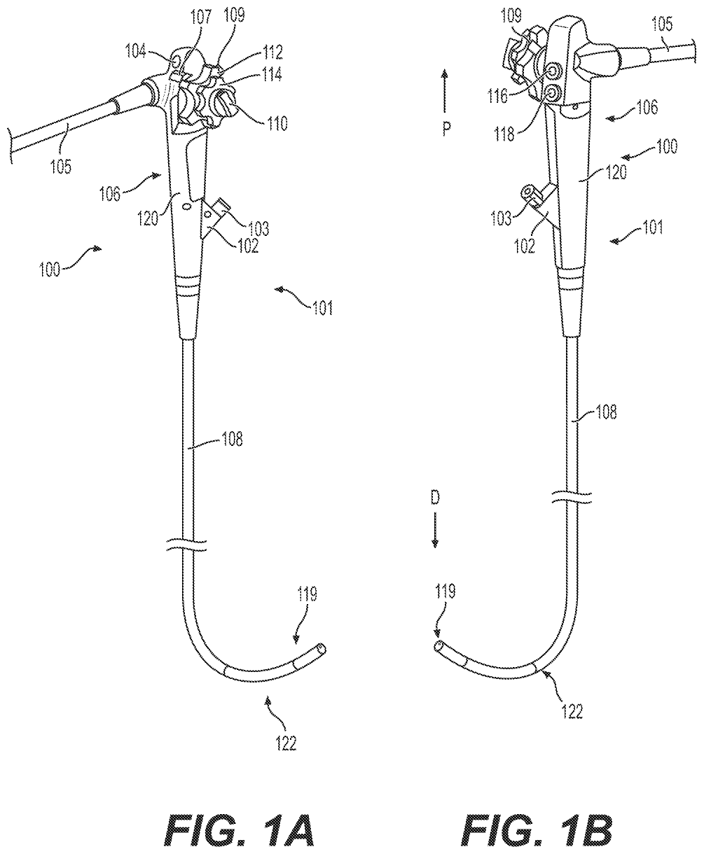

FIGS. 1A and 1B show a perspective view of an exemplary endoscope system 100. Endoscope system 100 may include an endoscope 101. Although the term endoscope may be used herein, it will be appreciated that other devices, including, but not limited to, duodenoscopes, colonoscopes, ureteroscopes, bronchoscopes, laparoscopes, sheaths, catheters, or any other suitable delivery device or medical device may be used in connection with the devices of this disclosure, and the connector assembly discussed below may be incorporated into any of these or other medical devices. Endoscope 101 may include a handle assembly 106 and a flexible tubular shaft 108. The handle assembly 106 may include a biopsy port 102, a biopsy cap 103, an image capture button 104, an elevator actuator 107, a first locking lever 109, a second locking lever 110, a first control knob 112, a second control knob 114, a suction button 116, an air/water button 118, a handle body 120, and an umbilicus 105. All of the actuators, elevators, knobs, buttons, levers, ports, or caps of endoscope system 100, such as those enumerated above, may serve any purpose and are not limited by any particular use that may be implied by the respective naming of each component used herein. The umbilicus 105 may extend from handle body 120 to auxiliary devices, such as a control unit, water/fluid supply, and/or vacuum source. Umbilicus 105 therefore may transmit signals between endoscope 101 and the control unit, in order to control lighting and imaging components of endoscope 101 and/or receive image data from endoscope 101. Umbilicus 105 also can provide fluid for irrigation from the water/fluid supply and/or suction to a distal tip 119 of shaft 108. Buttons 116 and 118 control valves for suction and fluid supply (e.g., air and water), respectively. Shaft 108 may terminate at a distal tip 119. Shaft 108 may include an articulation section 122 for deflecting distal tip 119 in up, down, left, and/or right directions. Knobs 112 and 114 may be used for controlling such deflection, and locking levers 109 and 110 may lock knobs 112 and 114, respectively, in desired positions. Handle body 120 may be tapered and may narrow as the handle extends distally such that the profile of the handle body 120 is smaller at its distal end than at its proximal end.

Distal tip 119 may include an imaging device (e.g., a camera) and a lighting source (e.g., an LED or an optical fiber). Distal tip 119 may be side-facing. That is, the imaging device and the lighting source may face radially outward, perpendicularly, approximately perpendicularly, or otherwise transverse to a longitudinal axis of shaft 108 and distal tip 119. In alternatives, distal tip 119 may be forward-facing. That is, the imaging device and the lighting device may face approximately along a longitudinal axis of distal tip 119 and shaft 108.

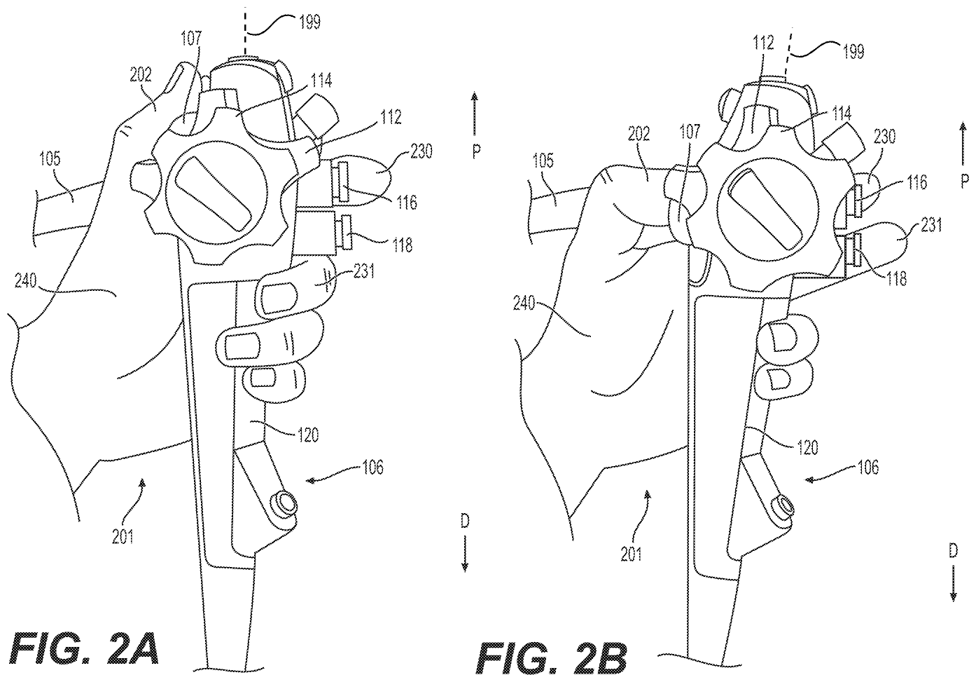

In operating endoscope system 100, a user may use his/her left hand to hold the handle assembly 106 (shown in FIG. 2A) while the right hand is used to hold accessory devices and/or operate one or more of the actuators of the handle assembly 106, such as the first and second control knobs 112, 114 and the first and second locking levers 109, 110. The user may grasp the handle assembly 106 by wrapping the user's hand around the handle body 120. When grasping handle body 120, the user may use the left thumb to operate the first and second control knobs 112, 114 and the elevator actuator 107 (through rotation about their respective axes), and may use a left-hand finger to operate the image capture button 104, the suction button 116, and/or the air/water button 118 (each by pressing). The user may rotate the handle assembly 106 in order to rotate shaft 108 about a longitudinal axis of shaft 108 and position distal tip 119 at a target area within a patient's body.

FIGS. 2A and 2B show an exemplary user's left hand 201 grasping handle assembly 106, which may have a central longitudinal axis 199 extending longitudinally through handle assembly 106. The user's left index finger 230 and middle finger 231 may be used to operate the suction button 116 and the air/water button 118. The user may position the thumb 202 of the left hand 201 (the grasping hand) over the elevator actuator 107 and move the elevator actuator 107 along a circular path from a first position (shown in FIG. 2A) to a second position (shown in FIG. 2B) by moving the thumb 202. As shown in FIG. 2B, the palm 240 may move away from the handle body 120 when the thumb 202 moves from the first position to the second position. For some conventional endoscopes, when a user's hand is contorted and/or the user's wrist is bent, actuation of the actuators 107, 112, 114, 116, 118 on handle assembly 106 may be more difficult and may increase the strain on a user's hand, wrist, and fingers. The connector assembly discussed herein below may reduce the strain caused by awkward positioning of the user's hand, facilitate actuation of actuators 107, 112, 114, 116, 118, and alleviate wrist pain caused by excessive bending of the wrist during operation of some conventional endoscopes.

Furthermore, for some conventional endoscopes, users may have to strain their wrists and/or awkwardly contort their hands in order to position shaft 108 and distal tip 119 at an appropriate position within the patient's body, such as when a camera at the distal tip 119 is facing a target area. For example, with some conventional endoscopes, the users may rotate handle assembly 106, producing an awkward angle for gripping and operating elements of handle assembly 106. The connector assembly discussed herein may also facilitate rotation of shaft 108 and positioning of distal tip 119 during operation of some conventional endoscopes, thereby reducing and/or alleviating strain to a user's wrist, fingers, and/or hand.

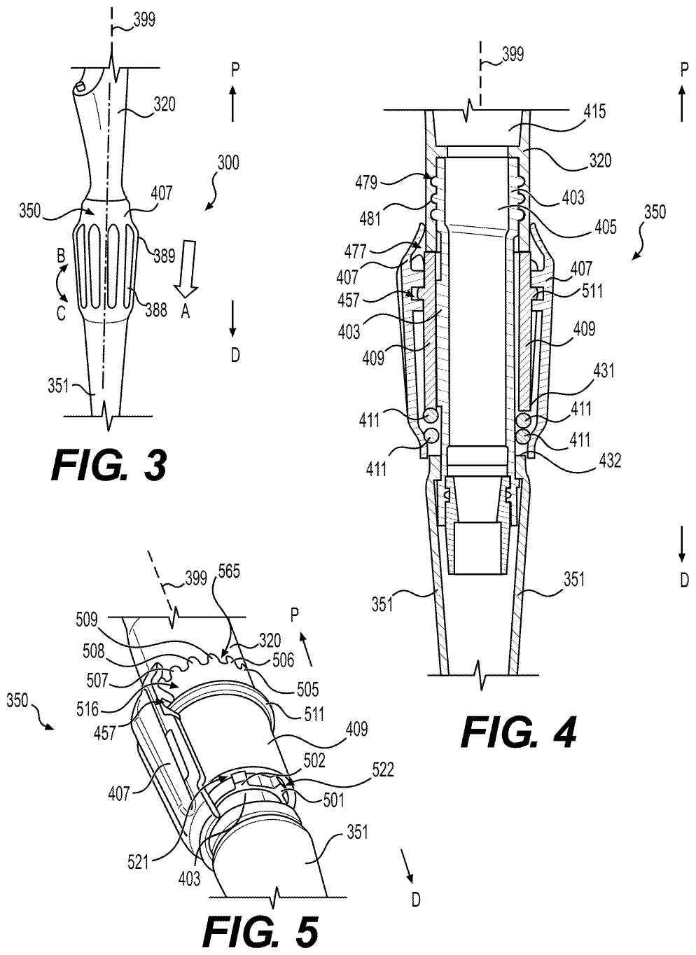

FIG. 3 illustrates a side view of a portion of an endoscope 300, including a connector assembly 350 coupling a shaft portion 351 to a handle portion 320 of the endoscope 300. Endoscope 300 may have any of the features discussed hereinabove with relation to endoscope 101. Connector assembly 350 may allow a user to rotate shaft 351 about central longitudinal axis 399 relative to handle 320, as shown by directions arrows B, C in FIG. 3. Specifically, connector assembly 350 may fixedly couple handle 320 to shaft 351 when in a neutral position, as shown in FIG. 3. To rotate shaft 351 relative to handle 320, a user may push knob portion 407 of connector assembly 350 distally (shown as arrow A in FIG. 3) to transition connector assembly form the neutral position to an engaged position in which the user may rotate shaft 351 about axis 399 relative to handle 320. While pushing distally on knob portion 407 (with connector assembly 350 in the engaged position), the user may rotate shaft 351 freely about axis 399 relative to handle 320. Handle 320 and shaft 351 remain rotatably coupled together via connector assembly 350 when connector assembly 350 is in the engaged position. When the user releases knob portion 407, connector assembly 350 may then transition to the neutral position in which handle 320 is fixedly coupled to shaft 351. In other words, shaft 351 may not rotate relative to handle 320 in the neutral position. In some examples, connector assembly 350 may be biased towards the neutral position. In other, alternative, examples, connector assembly 350 may be biased towards the engaged position. In such alternative examples, a user would push proximally on knob portion 407 to transition the connector assembly 350 to the neutral position and would release knob portion 407 to transition the connector assembly to the engaged position. The operation and structure of connector assembly will be discussed in more detail herein below with regard to FIGS. 8A-8D.

FIG. 4 illustrates a side cross-sectional view of connector assembly 350. As shown in FIG. 4, connector assembly 350 includes knob portion 407 at a radially-outermost portion, relative to central longitudinal axis 399, of connector assembly 350. Knob portion 407 may be approximately cylindrical and may include a central, longitudinal lumen 477 extending longitudinally through knob portion 407. Knob portion 407 may include tapered proximal and distal ends. Knob portion 407 may be configured to receive a distal portion of handle 320 and a proximal portion of shaft 351. A series of protrusions 389 and recesses 388 may extend longitudinally on a radially-outer surface, relative to axis 399, of knob portion 407. The series of protrusions 389 and recesses 388 may be spaced circumferentially around knob portion 407 and about axis 399. The series of protrusions 389 and recesses 388 may be configured to facilitate rotation of knob portion 407, provide grip, and facilitate actuation connector assembly 350 via a user's hand, fingers, and/or thumb.

A collar 409 is positioned between handle 320 and shaft 351. A radially-outer surface, relative to axis 399, of collar 409 abuts a radially-inward facing surface, relative to axis 399, of knob portion 407. Collar 409 includes a protrusion 511 extending circumferentially around collar 409, and protrusion 511 is received within a recess 457 of knob portion 407. Protrusion 511 may be configured to restrict radial and linear movement between knob portion 407 and collar 409 such that knob portion 407 and collar 409 move integrally with each other (i.e., as a single component). Collar 409 includes teeth 505-509 (shown in FIG. 5) extending circumferentially about axis 399 at the proximalmost end of collar 409, and teeth 505-509 engage with complimentary teeth 565 at a distal end portion of handle 320. Although only teeth 505-509 are shown in FIG. 5, teeth 505-509 may extend circumferentially about axis 399, may be evenly spaced from adjacent teeth 505-509, and may extend entirely around axis 399. A spring 411 is positioned within the central lumen 477 between collar 409 and shaft 351, and may extend circumferentially about axis 399 and sleeve 403. A proximalmost end of spring 411 may abut a distal-facing surface of collar 409, and a distalmost end of spring 411 may abut a proximal-facing surface 432 of shaft 351. Spring 411 may exert a proximal force on collar 409, pushing it towards handle 320. Spring 411 may be replaced with any other biasing member known in the art, such as a shape memory material body or other resilient biasing means.

A sleeve 403 may be fixedly coupled to a proximal end portion of shaft 351 and may be rotatably coupled to handle 320. Sleeve 403 may be approximately cylindrical and may include a central lumen 405 extending longitudinally through sleeve 403. Sleeve 403 may extend through a central lumen of collar 409, and a radially-outer surface of sleeve 403, relative to axis 399, may abut a radially-inward facing surface of collar 409. In some examples, sleeve 403 may include one or more protrusions 479 extending radially-outward relative to axis 399, and the one or more protrusions 479 may be received within recesses 481 of handle 320. The one or more protrusions 479 may allow sleeve to rotate about axis 399 relative to handle 320, but may not move proximally or distally relative to handle 320. Sleeve 403 is coupled to collar 409 in such a way that collar 409 may move freely relative to sleeve 403 in the proximal and distal directions, and rotation of collar 409 about axis 399 may also rotate sleeve 403 about axis 399. Sleeve 403 may be fixedly coupled to shaft 351 such that rotation of sleeve 403 also rotates shaft 351. Sleeve 403 may be metal, plastic, and/or any other suitable material known in the art.

FIG. 5 illustrates a perspective view of connector assembly 350 with a portion of the knob portion 407 removed to expose interior components of connector assembly 350. Note that spring 411 is removed from FIG. 5 to more clearly illustrate other components of connector assembly 350. Teeth 505-509 of collar 409 are shown received by corresponding teeth 565 of handle 320, and protrusion 511 is shown positioned within recess 457. Sleeve 403 may include ribs 501, 502 extending longitudinally along a radially-outer surface of sleeve 403. Ribs 501, 502 may be received by recesses 521, 522 in collar 409. Each rib 501, 502 is received by a corresponding recesses 521, 522, and any number of ribs and recesses may be included in sleeve 403 and collar 409. Ribs 501, 502 and recesses 521, 522 may facilitate linear motion of collar 409 in the proximal and distal directions relative to sleeve 403, and may prevent rotation of collar 409 about axis 399 relative to sleeve 403.

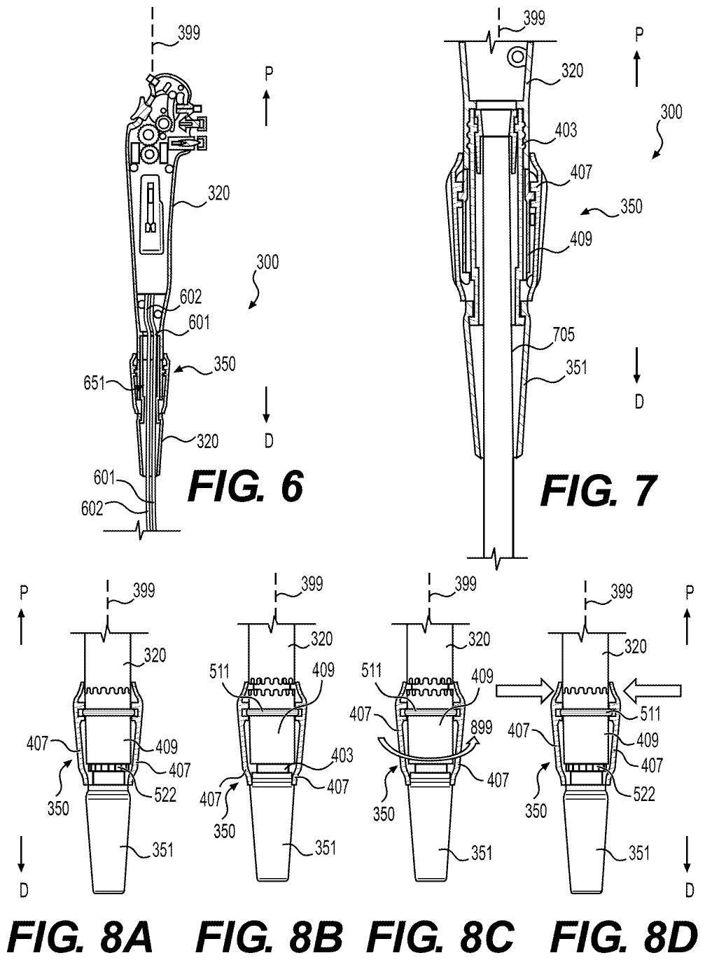

FIG. 6 illustrates a side, cross-sectional view of endoscope 300 including handle 320, connector assembly 350, and shaft 320. A portion of shaft 320 is removed in FIG. 6. Internal cables 601, 602 are shown extending from an interior portion of handle 320, through connector assembly 350, and through shaft 320. Internal cables 601, 602 may be positioned within a central longitudinal lumen 651 of connector assembly 350. In some examples, central longitudinal lumen 651 may include central lumen 405 of sleeve 403. Internal cables 601, 602 may freely move within and through connector assembly 350, and connector assembly 350 may rotate without moving internal cables 601, 602. Internal cables 601, 602 may include electrical wiring, articulation wires and/or cables, elevator actuating cables, actuating cables for any device positioned proximal to handle 320, and/or any other type of cable, wire, or other body known in the art, which may include elements to control aspects of distal tip 119.

FIG. 7 illustrates a side cross-sectional view of connector assembly 350, handle 320, and shaft 351. In the connector assembly 350 shown in FIG. 7, a tube 705 extends from a proximal portion of handle 320, through connector assembly 350, and forms a portion of shaft 351. Tube 705 is not shown in cross-section in FIG. 7. Tube 705 may be approximately cylindrical and may include a central longitudinal lumen extending the length of tube 705. Tube 705 may be fixedly coupled to sleeve 403 at a proximal end portion of tube 705. Tube 705 may rotate as sleeve 403, collar 409, and knob portion 407 rotates about axis 399. Tube 705 may be configured to receive internal cables 601, 602, and may extend to a distal end portion of endoscope 300. In other examples, connector assembly 350 may not include tube 705. Tube 705 may be flexible, and may be made of any suitable flexible, biocompatible material known in the art.

In operation of endoscope 300, connector assembly 350 may allow a user to rotate shaft 351 relative to handle 320 without adjusting the user's grip on handle 320, for example without contorting the user's wrist. FIGS. 8A-8D illustrate various positions of connector assembly 350 as a user operates connector assembly 350 to rotate shaft 351 relative to handle 320. Note spring 411 is removed from FIGS. 8A-8D purely for illustration purposes. In FIG. 8A, connector assembly 350 is shown in a neutral position in which shaft 351 is coupled to handle 320 such that rotation of handle 320 causes rotation of shaft 351. In the neutral position, teeth 505-509 of collar 409 are engaged with teeth 565 of handle 320 preventing the rotation of collar 409 relative to handle 320. A user is not pushing knob portion 407 proximally when connector assembly 350 is in the neutral position.

For a user to rotate shaft 351 about axis 399 relative to handle 320, the user first grasps knob portion 407 and pushes knob portion 407 distally. As shown in FIG. 8B, when a user pushes knob portion 407 distally collar 409 moves distally relative to handle 320. The user may compress spring 411 when the user moves knob portion 407 distally, and teeth 505-509 of collar 409 may become longitudinally spaced from teeth 565 of handle 320 (shown in FIG. 8B). Knob portion 407 pushes collar 409 distally relative to shaft 403 as the user pushes knob portion 407 distally. Once teeth 505-509 of collar 409 become longitudinally spaced from teeth 565 of handle 320, the user may rotate knob portion 407 relative to handle 320 in order to rotate collar 409, sleeve 403, and shaft 351 about axis 399 relative to handle 320. This rotation is shown in FIG. 8C via arrow 899. The user also maintains a distal pushing force on knob portion 407 while rotating knob portion 407 about axis 399 to prevent spring 411 from moving knob portion 407 and collar 409 proximally. The positions of connector assembly 350 shown in FIGS. 8B and 8C are engaged positions in which the user may rotate shaft 351 relative to handle 320. A radially-inward facing surface of collar 409 may slide across ribs 501, 502 of sleeve 403 when collar 409 moves proximally or distally.

Once the user has finished rotating shaft 351, the user may release knob portion 407 and spring 411 may push knob portion 407 and collar 409 proximally to re-engage teeth 505-509 of collar 409 with teeth 565 of handle 320. FIG. 8D illustrates connector assembly 350 returned to a neutral position after the user released knob portion 407, and shaft 351 may not be rotated about axis 399 relative to handle 320 once in the neutral position. As compared with the configuration of FIG. 8A, shaft 351 may be rotated relative to handle 320 in FIG. 8D.

Medical devices, such as endoscopes, connector assemblies, and handles of this disclosure may assist with ergonomically positioning a user's hand and wrist when the user operates endoscope 101 or other medical devices, may reduce hand strain caused by excessive movement and/or bending of the wrist when the user operates an endoscope 101, 300, and may reduce the chance of the user losing his or her grip. Also, the medical devices, connector assemblies, and handles may help prevent repeated repositioning of a user's hand on a medical device handle due to fatigue, strain, or other difficulty. Furthermore, medical devices, connector assemblies, and handles may help facilitate positioning of a distal end portion of a medical device without excessive hand strain due to rotation of a handle. Each of the aforementioned connector assemblies, whether used in conjunction with an endoscope system or any other medical device, may be used to enhance and/or facilitate a user's grip on a handle. Any portion of the connector assemblies discussed herein may be incorporated into a handle of an endoscope or other medical device to improve a user's operation of the device.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

We claim:

1. A connector assembly for a medical device, the connector assembly comprising:
a sleeve configured to fixedly couple to a shaft of the medical device and rotatably couple to a handle of the medical device;
a collar positioned around the sleeve and configured to couple to the sleeve such that collar (i) may move in a proximal and distal direction relative to sleeve and (ii) may rotate sleeve about a central longitudinal axis when the collar is rotated about the central longitudinal axis; and
a biasing member;
wherein the connector assembly is configured to transition between a neutral position and an engaged position.

2. The connector assembly of claim 1, wherein the knob portion and the collar cannot rotate relative to the handle in the neutral position; and
wherein the knob portion and the collar can rotate relative to the handle in the engaged position.

3. The connector assembly of claim 2, further comprising a knob portion positioned around the collar; wherein the knob portion includes ribs.

4. The connector assembly of claim 1, wherein the sleeve includes at least one protrusion at a proximal portion of the sleeve, and the at least one protrusion is configured to be received in a recess of the handle.

5. The connector assembly of claim 1, wherein the biasing member is a spring.

6. The connector assembly of claim 1, further comprising a tube extending through the collar.

7. The connector assembly of any of the preceding claims, wherein the sleeve includes a series of longitudinal ribs and the collar includes a series of longitudinal recesses configured to receive the longitudinal ribs.

8. The connector assembly of claim 7, wherein the longitudinal ribs are configured to move proximally and distally through the longitudinal recesses.

9. The connector assembly of claim 1, wherein the collar includes a protrusion extending circumferentially around a central longitudinal axis of the collar.

10. The connector assembly of claim 9, wherein the knob portion includes a recess extending circumferentially around a radially-inward facing surface of the knob portion, and wherein the recess is configured to receive the protrusion.

11. The connector assembly of claim 1, wherein each of the collar, the knob portion, and the sleeve is approximately cylindrical and includes a respective central longitudinal lumen.

12. The connector assembly of claim 1, wherein the collar includes a series of teeth extending proximally from a proximal end portion of the collar.

13. The connector assembly of claim 12, wherein the series of teeth are configured to be received by a complimentary series of teeth of the handle.

14. The connector assembly of claim 13, wherein the series of teeth of the collar are received by the complimentary series of teeth of the handle in the neutral position; and wherein the series of teeth of the collar are spaced from the complimentary series of teeth of the handle in the engaged position.

15. The connector assembly of claim 1, wherein the collar is fixedly coupled to the knob portion.

16. A medical device comprising:
a handle body;
a shaft extending longitudinally to a distal tip; and
connector assembly comprising:
a sleeve fixedly coupled to the shaft and rotatably coupled to a handle body;
a collar positioned around the sleeve and coupled to the sleeve such that collar (i) is movable in a proximal and distal direction relative to sleeve and (ii) is rotatable about a central longitudinal axis of the medical device during rotation of the collar about the central longitudinal axis;
a knob portion positioned around the collar; and
a biasing member;
wherein the connector assembly is configured to transition between a neutral position and an engaged position.

17. The medical device of claim 16, wherein the collar includes a series of teeth extending proximally from a proximal end portion of the collar; and wherein the series of teeth are configured to be received by a complimentary series of teeth of the handle body.

18. The medical device of claim 16, wherein the sleeve includes protrusions at a proximal portion of the sleeve, and the protrusions are configured to be received in recesses of the handle body.

19. A method of operating a medical device that includes a handle and a shaft extending longitudinally from the handle, the method comprising:
moving a knob portion of a connector assembly distally and compressing a biasing member of the connector assembly;
rotating the knob portion about a central longitudinal axis of the knob portion relative to the handle; and
releasing the knob portion to fixedly couple the shaft to the handle.

20. The method of claim 19, wherein moving the knob portion of the connector assembly distally includes moving teeth of a collar of the connector assembly away from teeth at a proximal end portion of the handle.
